(19) United States
(12) Patent Application Publication
(10) Pub. No.: US 2018/0125715 A1
(43) Pub. Date: May 10, 2018

(54) PROTECTIVE MASK

(71) Applicant: Dario Bellussi, Venezia (IT)

(72) Inventor: Dario Bellussi, Venezia (IT)

(21) Appl. No.: 15/574,859

(22) PCT Filed: May 18, 2016
(86) PCT No.: PCT/IB2016/052908

§ 371 Date: Nov. 17, 2017

(30) Foreign Application Priority Data
May 19, 2015 (IT) 102015000015930
Jun. 24, 2015 (IT) 102015000027278

(51) Int. Cl. A61F 9/02; G02B 25/00; G02C 7/08; G02C 3/00; G02B 7/00

(52) U.S. Cl. CPC A61F 9/029; G02B 25/004; G02B 7/002; G02C 3/003; G02C 7/088

(57) ABSTRACT

The magnifying system (5) of the protective mask (1) according to the invention occupies a first more central portion of the visual field of the user with a minimum focussing distance equal to or less than 10 meters. The screen (3) covers the eyes of the user allowing a more peripheral vision. The screen (3) and the sealing system, together with other possible components of the mask, form a casing that completely encloses and isolates from the external environment the user's eyes and the area of the face adjacent to the eyes, forming a barrier that prevents solid or liquid projections such as, for example, solid fragments, splashes, drops or aerosol particles to penetrate into the mask (1, 1′) and reach the user's eyes.

### FIELD OF THE INVENTION

The present invention relates to a protective mask particularly suitable for shielding the eyes of surgeons from contact with solid or liquid projections produced during surgical operations, and also able to provide a magnified vision of part of the field of operation and wide lateral vision on the rest of the visual field.

The masks according to the invention can have many other applications such as laboratory examinations, scientific research, artisanal works or any manufacturing activities in general.

### STATE OF THE ART

During a surgical operation, the doctor/surgeon or dentist has at the same time the need to visualize both the magnified vision of the anatomical part on which he is operating and to be able to control a wider area of the field of operation around the operated area or in the operating room through its peripheral vision; last but not least the doctor/surgeon or dentist must be properlyprotected from infections transmitted through the eye.

In order to meet the first two needs, in surgery it is known for a long time to use optical instruments which substantially combine the structure of common eyeglasses or sunglasses with magnifying optical systems of Galilean or prismatic type, providing a stronger magnification.

Examples of this kind of optical instruments are described in publications U.S. Pat. No. 2,226,941, U.S. Pat. No. 4,196,966 or U.S. Pat. No. 5,331,357.

This particular kind of instrument protects the surgeon very poorly from very dangerous and potentially fatal infections, including hepatitis B and C and HIV, transmissible through the eye.

The eye mucosa is extremely vascularized and directly exposed to the external environment and, unlike an uninjured epidermis, it does not constitute a mechanical barrier against penetration and consequent infection by etiological factors of these pathologies such as bacteria, mycetes, virus or prions.

If reached by splashes of blood, infected water, vomit or solid infected particles the eye mucosa can constitute an easy access for pathogenic microorganisms to reach the blood stream and cause an infection.

Infections transmissible through the eye are extremely dangerous, some of these can lead in the medium/long-term to the death of the infected operator; an analysis of the relevant scientific literature reveals the risk of developing acute or chronic hepatitis, cirrhosis or even worse, hepatocellular carcinoma following infection from the hepatitis B (HBV) or hepatitis C virus (HCV).

The same arguments hold for the HIV virus, responsible for AIDS (acquired immune deficiency syndrome): the literature documents the risk of seroconversion following infection, transmissible through the eye, from said etiological factor.

Cases of HBV, HCV or HIV infection through the eye are relatively common amongst health professionals.

It would therefore be desirable to reduce, if not totally eliminate, the risk of contracting infections through the eye, allowing health professionals to conduct their work in the best safety and serenity, securing their health and reducing costs for the National Sanitary Systems to cure these infections, such as the cost for post-exposure chemoprophylaxys, medical examinations, insurances and sick leaves.

Currently, the only method widely utilized in operating rooms for protection from infected splashes or particles is to wear masks with a wide transparent visor which covers the whole face, as described for example in publication US2012/047614A.

This particular mask cannot bear magnifying systems such as the ones described in publications U.S. Pat. No. 2,226,941, U.S. Pat. No. 4,196,966 and U.S. Pat. No. 5,331,357 and does not protect adequately from infections transmitted through the eye mucosa.

The author of the present invention has noticed several times that liquid drops or other infected particles, are projected at high speed—for example, in the case of a dentist, an oral and maxillofacial surgeon or an orthopaedic surgeon intervening on hard bodily tissues utilizing milling cutters mounted on high-speed micro-motors under continuous spray of air/water—these particles, moving in all directions towards the face of the surgeon, particularly in directions skimming across the face rather than perpendicularly to it, easily got behind the transparent visor and reached the eyes, causing infections or making it necessary for the surgeon to undergo preventive chemotherapy provided for in such cases by health and safety protocols, a procedure which reduces but does not eliminate the risk of developing a HBV, HCV or HIV infection after exposure.

Furthermore, the eyewear described, for example, in publications U.S. Pat. No. 2,226,941, U.S. Pat. No. 4,196,966 or U.S. Pat. No. 5,331,357 tends to fog up because of the humidity deviated towards the lenses from the mouth-covering surgical masks M in nonwoven fabric commonly used in surgical operations. The shape of the frame, basically identical to normal reading or sun glasses, makes them not very suitable for holding illuminating devices or video cameras, both of which need an electrical and memory autonomy of several hours; consequently their weight is such that its bridge and temples cause excessive and painful pressure on the rooth and dorsum of the nose, on the ears of the user as well as excessive and unbalanced annoying loads on the cervical vertebrae.

Consequently at present a surgeon or dentist who wishes to have magnified vision during a surgican operation is obliged to wear magnifying lenses such as those described in documents U.S. Pat. No. 2,226,941, U.S. Pat. No. 4,196,966 or U.S. Pat. No. 5,331,357, increasing the risk of contracting infections transmissible through the eye; the surgeon who wishes to be protected from infections therefore has to do without magnifying optical systems.

Publication U.S. Pat. No. 4,395,731 describes a medical television microscope, in which the casing 31 seems to completely cover the ocular region of the user preventing splashes and solid infected particles from reaching the eyes, although this aim or advantage is not explicitly mentioned.

However this device or mask does not allow peripheral vision of the field of operation and the user has a very restricted visual field with highly magnified vision; in order to have a non-magnified vision and non-obstructed vision of the field of operation, the document suggests removing the mask, a procedure that is wasteful of precious time, and which may prove to be impractical or that even cannot be considered during an operation.

Publication U.S. Pat. No. 1,033,965 describes a magnifying binocular system for artisans and scientists, inappropriate for surgical use as it strongly restricts the angle of the visual field substantially blocking peripheral vision.

Moreover the two large openings 5, meant to allow downwards vision, provide direct path for drops, splashes and infected particles towards the eyes, leaving them very scarcely protected.

Publications GB224338, U.S. Pat. No. 1,982,444, U.S. Pat. No. 2,024,322 and DE650682 describe glasses which completely enclose the area around the eyes allowing a certain degree of peripheral vision; being designed for military use, for pilots or drivers, the binoculars need to ensure magnified vision of very distant objects, situated at least several dozen meters away and are therefore clearly inappropriate for surgeons who need to have a magnified vision of much closer objects.

One aim of the present invention is to solve the aforementioned problems which are present in the current state of the art and in particular to provide a magnifying optical system, useful for surgeons and dentists to have a magnified vision of the area in which they are operating, which better protects the zone around the eye from infections and which still allows a good peripheral view of the field of operation and, in general, of the surrounding environment.

### SUMMARY OF THE INVENTION

Such an aim is reached, according to the present invention, with a protective mask having characteristics according to claim 1.

Further characteristics of the device are object of the dependent claims.

The attainable advantages of the present invention will become clearer to the skilled person from the following detailed descriptions of some particular and non-limiting embodiments, described with reference to the following schematic figures.

### LIST OF FIGURES

FIG. 1 shows a perspective view of the protective mask worn by a user according to a first particular embodiment of the invention;

FIG. 2 shows a first lateral view of the mask in FIG. 1;

FIGS. 3 and 3BIS show a view from above and a perspective view from below of the mask in FIG. 1 respectively;

FIG. 4 shows a partial section of the area of the frame and the seal of the mask in FIG. 1, along the IV-IV section plane;

FIG. 5 shows a partial section of the area of the frame and the air vents of the mask in FIG. 1, along the V-V section plane;

FIG. 5BIS shows a partial section of the frame and the air vents of a protective mask according to a second particular embodiment of the invention;

FIG. 6 shows a front view of the screen of the mask in FIG. 1 worn by a user, FIG. 7 shows a perspective view of the protective mask according to a third particular embodiment of the invention;

FIG. 8 shows a second side view, partially in section, of the mask in FIG. 1, worn by a user;

FIG. 9 shows a partial section, along the IX-IX section plane, of the area that rests on the cheekbones, the frame and the seal of the protective mask according to a fourth particular embodiment of the invention;

FIG. 10 shows a side view of the protective mask and the supply kit according to a fourth particular embodiment of the invention worn by a user;

FIG. 11 shows a perspective view of the corrective eyepiece of the supply kit in FIG. 10.

### DETAILED DESCRIPTION

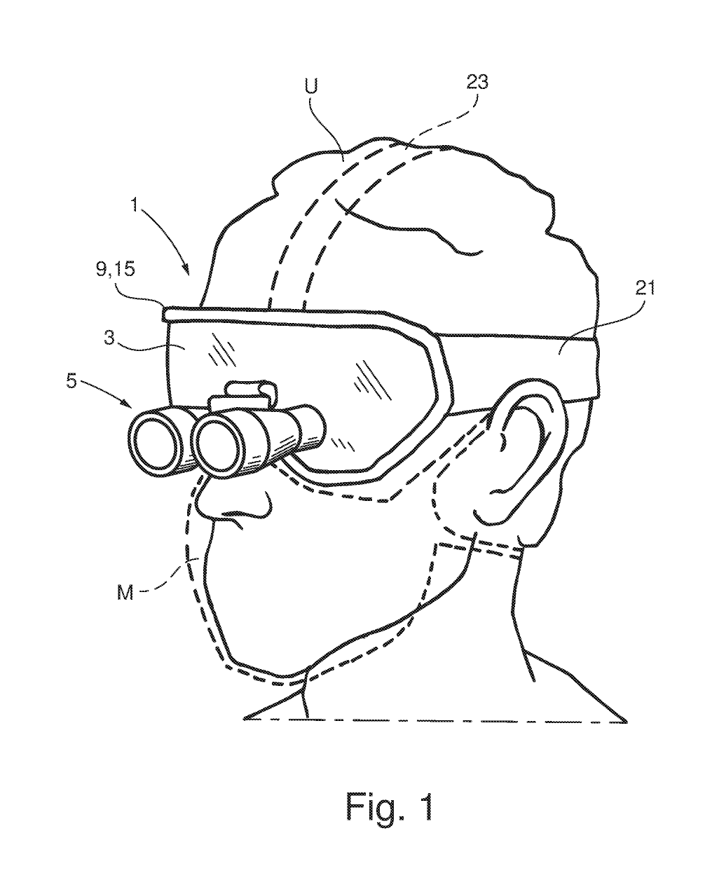

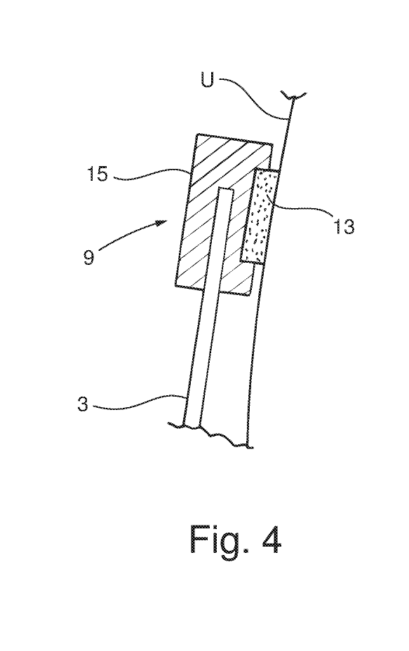

FIGS. 1-6, 8 show a protective mask according to a first particular embodiment of the invention, indicated with the overall reference 1.

Mask 1 contains:
a substantially transparent screen 3;
a magnifying optical system 5;
a fixing system, such as the support 7, which maintains the magnifying system 5 in a fixed position relative to the screen 3;
a sealing system 9.

According to an aspect of the invention, when a user U wears the mask 1:
the magnifying system 5 is configured for magnifying the images observed by the user occupying a more central first part of the visual field of the user with a minimum focussing distance not greater than 10 meters;
the screen 3 is configured for covering the eyes of the user allowing vision of a second portion of his visual field more peripheral in respect to the first more central portion of the visual field;
the sealing system 9 is configured for resting against the face of the user forming a substantially continuous barrier that surrounds or extends around the screen 3 and the eyes of the user;
the screen 3 and the sealing system 9, together with other potential elements of the mask 1, form a preferably box-like casing that completely encloses and isolates from the external environment the user's eyes and the area of the face adjacent to the eyes, forming a mechanical barrier that prevents solid or liquid projections such as, for example, solid fragments, splashes, drops or infected or damaging aerosol particles to penetrate into the mask and reach the user's eyes.

The magnifying optical system 5 preferably comprises a binocular system in its turn comprising a pair of telescopes, or rather monoculars, 11 of Galilean or prismatic type (FIGS. 1, 2, 3, 7).

Galilean telescope magnifying systems have the advantage of a greater depth of field of view at equal magnification, a simpler construction, and a corresponding lower price when compared to prismatic systems.

These latter are not only heavier and bulkier compared to Galilean systems but also require a relatively long, troublesome and user-dependant period of visual training which is not necessary with Galilean systems; on the other hand, they may achieve a greater magnification when compared to Galilean systems.

Galilean or prismatic telescopes 11 or, in general, magnifying systems 5, have a minimum focussing range preferably not greater than 5 meters, preferably not greater than 1 meter or even more preferably not greater than 0.8-0.9 meters.

Preferably the minimum focussing range is comprised between 0.1-0.8 meters or between 0.3-0.7 meters, in order to have a good magnified vision of the operating table or the work bench on which the user is working with his hands.

The system 5 can give from 2× up to 20× optical magnifications, sufficient for the majority of surgical uses and able to magnify without losing perception of the surroundings, in medical jargon “without losing the operating field”; preferably the optical magnification would be between 2× and 6λ, more preferably between 2.5-5×.

The fixing system 7 positions and holds in place the magnifying optical system 5, preferably on the outside of the screen 3, in front of it.

It could comprise a bracket fixed on the frame 15 or directly onto the screen 3.

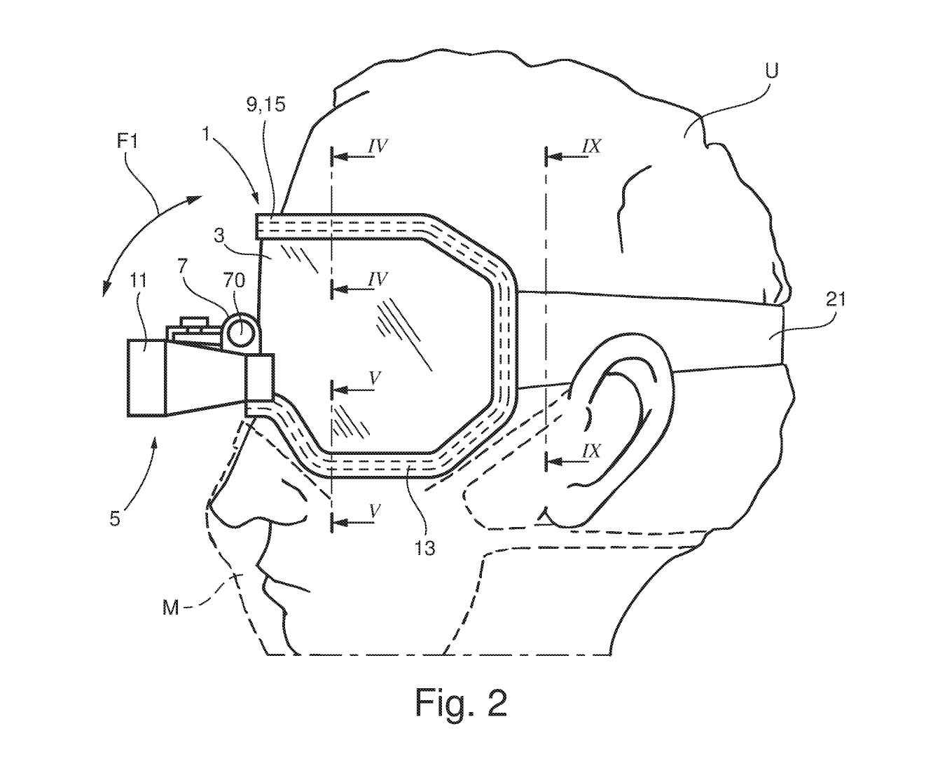

Ideally it could be provided with a hinge or other linkage 70 which allows twisting of the optical system of between, for example, 80° and 180° removing it from its position in front of the pupils of the user in case of need of non-magnified vision directly ahead (arrow F1 in FIG. 2).

The screen 3 can comprise, for example, of a glass plate or shell or, more preferably, of an appropriate transparent plastic material, such as polycarbonate, plexiglas or polypropylene; it can be free from any magnifying or demagnifying power, for example by having unvaried thickness, or having itself magnifying power, although preferably inferior to that of the magnifying optical system 5; for this reason the screen 3 can form a lens with the same corrective power as ordinary eyeglasses or reading glasses for users who wear corrective glasses in everyday life but don't want or can't wear them whilst wearing the mask 1.

The screen 3 is preferably polarized.

In order to have correcting power, the screen 3 could have a non uniform thickness in order to form itself a lens or comprise a transparent plate or shell of substantially uniform thickness, free of magnifying or reducing power, on which one or two corrective lenses are fused, glued, soldered or in some other way fixed, said lenses being for example made out of a flexible synthetic material. In this way, the screen 3 will allow the user to conserve a wide field of view and to have a vision, at least peripheral, of a majorpart or the entirety of the field of operation; furthermore, to observe non magnified objects it is sufficient to look at them through the screen 3 moving the pupils away from the magnifying ocular system 5.

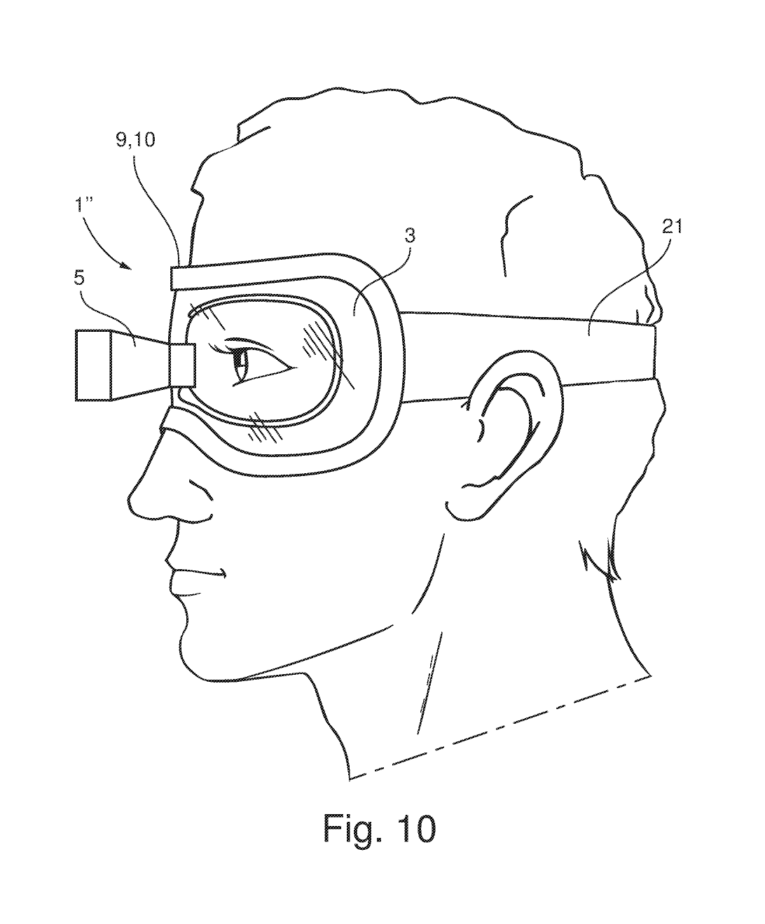

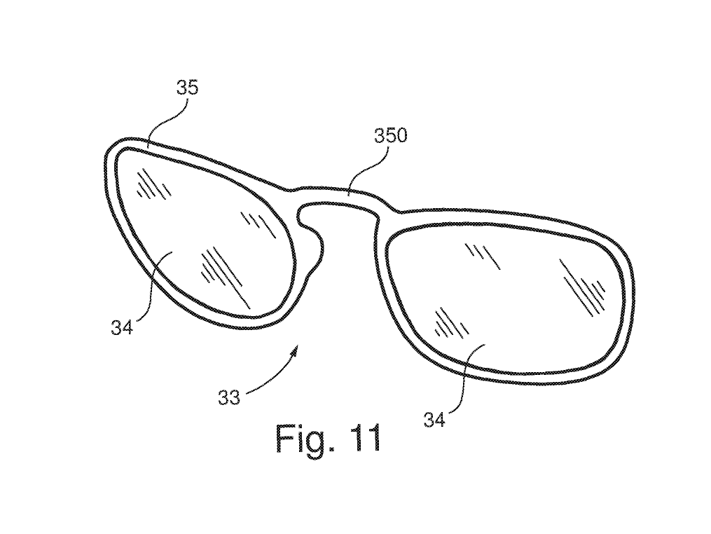

In a further particular embodiment shown in FIGS. 10, 11 the mask 1″ can be equipped with a suitable hooking zone that allows fixing of suitable corrective glasses 33 without temples, as described, for example, in document U.S. Pat. No. 8,550,620, in particular as shown in FIG. 20 of such document.

The corrective glasses without temples 33, in the present description also called “optical corrector 33”, can comprise two lenses 34 with corrective power and a frame 35 comprising in its turn a bridge 350.

The frame 35 may be fitted with rims 352 that partially or completely surround the lenses 34.

The optical corrector 33 is configured for locking on, or being fixed to, the mask 1″ in such a way that the lenses 34 are each positioned in front of one of the user's eyes and the bridge 350 extends above the nose of the user and between the lenses 34.

Preferably the optical corrector 33 is configured for being fixed to an area of the mask 1″ corresponding to or near to the nose of the user when he is normally wearing the mask; for example the optical corrector 33 can be configured for fitting into the indentation of the mask configured for accommodating the nasal bridge. Correspondingly the mask 1″ can be provided with suitable clasps to fix in place the optical corrector 33.

With this solution the protective mask according to the invention allow the most diverse vision defects be corrected maintaining very low production costs.

The mask 1″ and the corrector 33 could be sold together as a complete kit or bought separately.

In order to better adapt to the shape of the face and of the head of the user, the screen 3 is preferably curved at least in one spatial direction and possibly in two spatial directions to form a double curved shell.

In order to permit a wide peripheral vision and to allow the user to wear the mask 1 over ordinary eyeglasses, the screen 3 has an extension that allows the field of vision of the two eyes of the user to expand, in reference to the anatomical position of the body of the user:
by at least 120°, preferably by at least 140° but ideally by at least 160° measured on a transversal plane; and/or
by at least 80°, preferably by at least 100° but ideally by at least 120° measured on a sagittal plane.

To this purpose the screen 3 is relatively wide and preferably extends over a surface area equal or greater than 50 square centimetres, preferably equal or greater than 80 square centimetres, more preferably equal or greater than 120 square centimetres, more preferably equal or greater than 135 square centimetres even more preferably equal or greater than 155 square centimetres and most preferably equal or greater than 175 square centimetres.

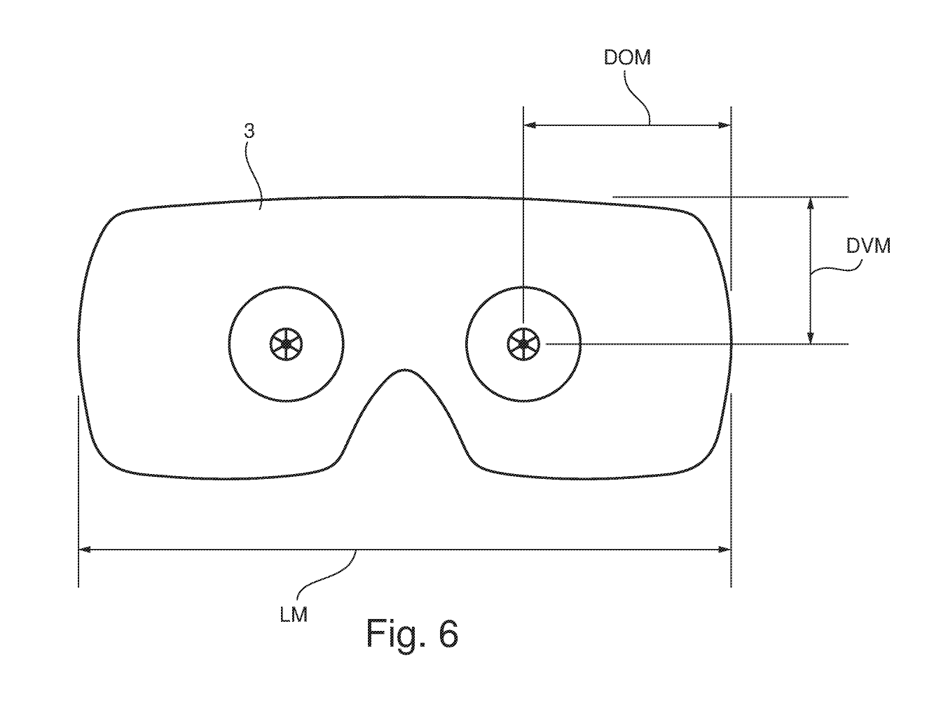

To this purpose the screen 3 has a width such that:
the minimum vertical distance DVM of the perimetral edges of the screen from the axis of the user's pupils measured on a transversal plane, when the user is looking straight ahead, is preferably equal or greater than 2 cm, more preferably equal or greater than 2.5-3 cm, most preferably equal or greater than 5 cm (FIG. 6); and/or
the minimum horizontal distance DOM of the perimetral edges of the screen 3 from the axis of the user's pupils measured on a transversal plane, when the user is looking straight ahead, is preferably equal or greater than 4 cm, more preferably equal or greater than 5 cm, most preferably equal or greater than 8 cm.

To this purpose, the surface of the screen 3 has an arc length LM preferably equal or greater than 12 cm, more preferably equal or greater than 15 cm, most preferably equal or greater than 18 cm.

In any case the transparent screen 3 extends around the eyepiece or eyepieces of the magnifying system 5.

The sealing system 9 can include a support seal 13 or a pad of substantially soft material, such as foam rubber or other natural or synthetic expanded elastic resin, or an appropriate elastic or fibrous material which preferably completely and substantially surrounds, without any interruptions, the screen 3 and the eyes of the user U, or surrounds or extends along at least 50%, more preferably along at least 80% but most preferably along at least 90% of the length of the perimeter of the screen 3 (FIGS. 2, 3, 4, 5).

The expanded material of the support seal 13 is preferably a closed cell material.

The support seal 13 preferably has the shape of a band or chord; it is configured for resting against the face of the user adapting to its shape. More specifically, the seal 13 is preferably fitted to include the superciliary arches and orbits of the user, to bypass the nose, cross the cheekbones or enclose them within the mask 1.

In case the seal 13 can enclose the nose inside the mask, for example passing over the jawbone.

The screen 3 would also have the appropriate shape and dimensions to extend over the superciliary arches, the orbits, at least part of the nose, the cheekbones and possibly also over part of the jawbone of the user. Preferably the screen 3 is configured for extending over the user's nose root.

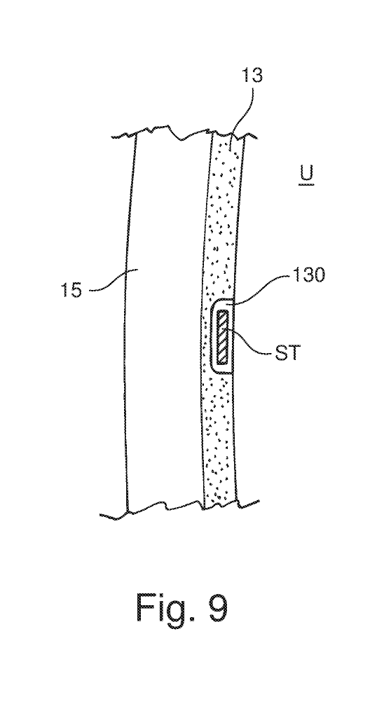

Preferably the support seal 13 is configured for resting against the face of the user along at least 80% of the perimeter of the screen 3, and more preferably along at least 90% of the perimeter of the screen 3, allowing to obtain the cut out zones 130, later described (FIG. 9).

The seal 13 can clearly rest against the face of the user along the whole perimeter of the screen 3.

Preferably the sealing system 9 includes a frame 15, which surrounds, for example completely, the screen 3 and on which the seal 13 is lodged.

The frame 15 is preferably made of a non-expanded and substantially rigid material, such as ABS, polypropylene (PP) or other appropriate non-expanded synthetic resin, suitable to maintain in position and confer a precise shape to the seal 9.

Preferably even the frame 15 has such a shape that would substantially match the face of the user U like a cast, for example on the forehead and on the area of the cheekbones and of the nose.

For this reason, the frame 15, and more generally the whole mask 1, 1′, can be produced as a unique specimen which fits the features of the user's face, obtained, for example, by laser-scanner, casts or image elaboration systems that can reconstruct the three-dimensional shape of the user's face from multiple bi-dimensional photographs.

The frame 15 may contain a first component 152 and a second component 154 (FIG. 7); on the second component 154 the seal 13 is mounted and on one side of the first component 152 the screen 3 is fixed, whilst on the other side the second component 154 is fixed. The second frame component 154 can be advantageously manufactured with rapid prototyping techniques such as 3D printing or stereolithography, using elastomeric or any materials that are softer than those used for component 152, in such a way to best adapt to the user's face and uniformly distribute contact pressure.

The first component 152 can be manufactured through injection moulding, potentially in a more rigid and resistant plastic material compared to the material used in component 154.

The frame 15, or more generally the mask 1, is advantageously equipped with a ventilation system configured for preventing or at least limiting fogging up of the internal part of the screen 3.

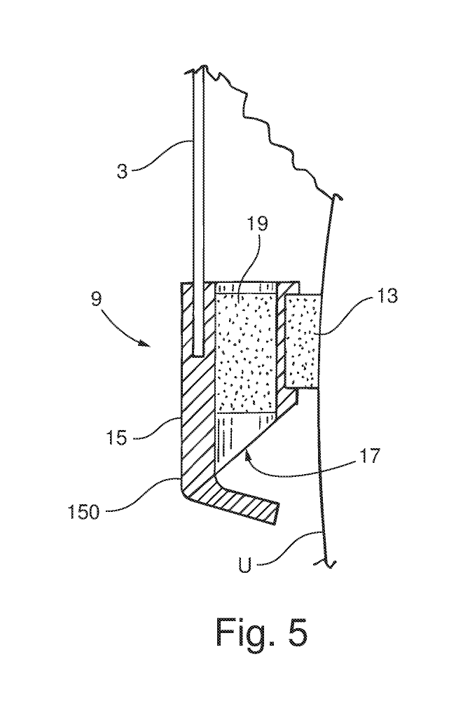

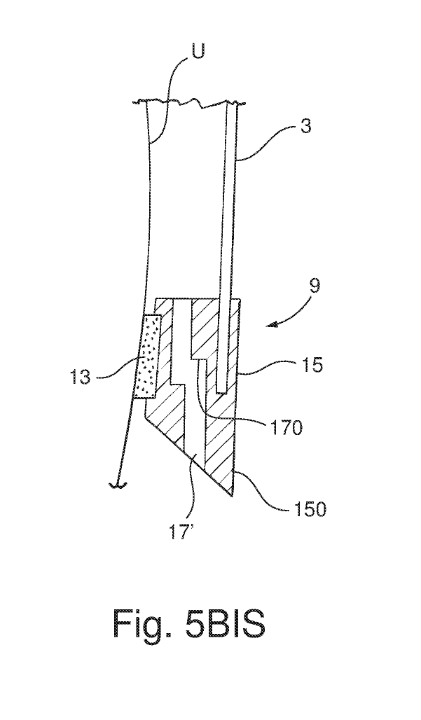

Such a ventilation system can include, for example, one or more air vents 17 closed for example by filters 19 made out of a suitable expanded material—such as, for example, a spongious open-cell structured material (FIG. 5)—, fibrous or granular, such as, an activated carbon bed or labyrinth or serpentine filters (FIG. 5BIS), which form a barrier permeable to air but able to trap granules, drops, splashes and other solid or liquid infected projections or contaminated projections such as drops or splashes of saliva, blood or vomit as well as fragments of bones, teeth or soft tissues (FIG. 5).

Serpentine filters 17′ can form passages with transversal sections, along section planes perpendicular to the axis of the frame 15 and to the surface of the user's face, with substantially a Z, zigzag or double bend shape (FIG. 5BIS); clearly in other particular embodiments they could have just an L shape forming, for example, a sharp edged bend. The solid or liquid particles that enter the opening 17′ and impact the step 170 will stop or in any case lose kinetic energy without reaching the eyes of the user.

In any case the screen 3, the support seal 13, the frame 15 and the other parts of the mask 1, as opposed to, for example, the binocular magnifier described in publication U.S. Pat. No. 1,033,965, do not have any openings or passages which would allow solid fragments, liquid splashes, droplets or aerosol particles to penetrate the inside of the mask and reach the eyes of the user.

Advantageously, as a further precaution, the transversal sections of the frame 15 form a longitudinal shelter 150 which shields and protects the entry to the air vents 17 from solid or liquid particles that hit the mask 1 in directions substantially perpendicular to the surface of the face and/or to the external surface of the screen 3 (FIG. 5,5BIS).

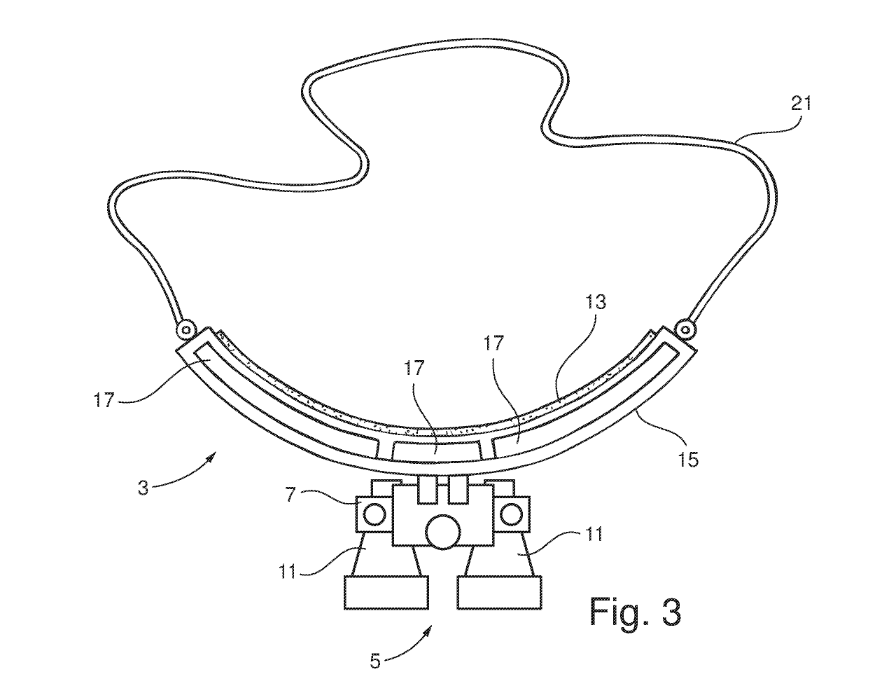

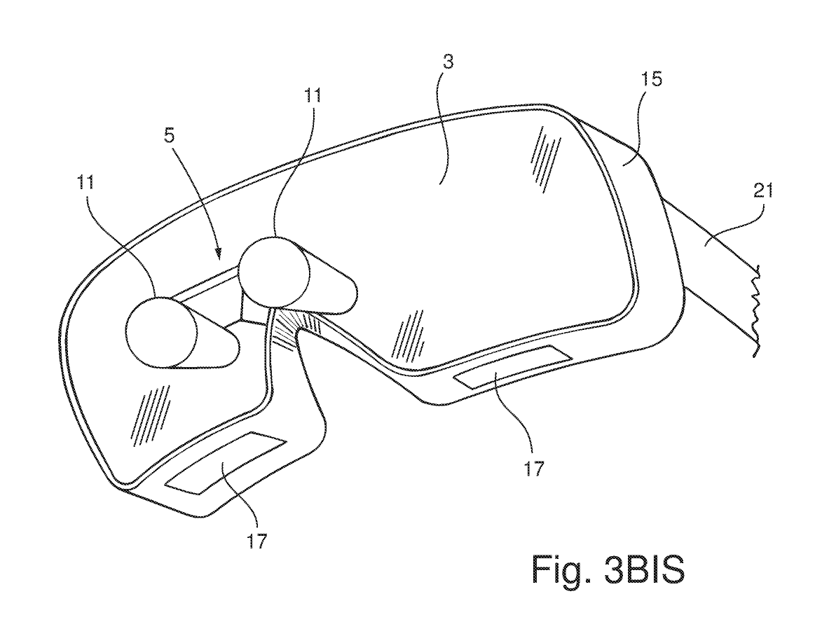

The air vent or vents 17 can be located in correspondence with the lower (FIG. 3BIS) or upper (FIG. 3) side of the mask 1, 1′, in proximity of the user's cheekbones or forehead, respectively.

In order to reduce fogging up of the inside of the screen 3, the seal 13, the frame 15, and more generally the mask 1, 1′ are advantageously shaped not to include the nostrils and the mouth of the user 1, preventing him from breathing into the mask (FIGS. 1, 2).

In order to secure it to user's head, the mask 1 can be equipped with a suitable elastic side strap 21 which can be extended in a substantially horizontal direction when the user's head is straight and passed over or near the ears; in addition to the strap 21 the mask can be equipped with further fixing straps, such as strap 23 which passes over the head from front to back (FIGS. 1, 2).

In order to allow the user to wear corrective glasses underneath the mask 3, on the support seal 13 and/or on the frame 15—for example on the two side areas—and/or on the screen 3 two or more cut-out areas and/or holes, not shown, can be arranged through which the temples of the glasses can exit to reach the ears in the normal way.

Alternatively the seal 13 can be thinned in certain zones to create one or more cut-outs 130 through which the temples ST of the glasses can exit (FIG. 9).

These openings could be closed by flexible membranes made of, for example, neoprene and provided with a central cut through which the temples of the glasses can pass.

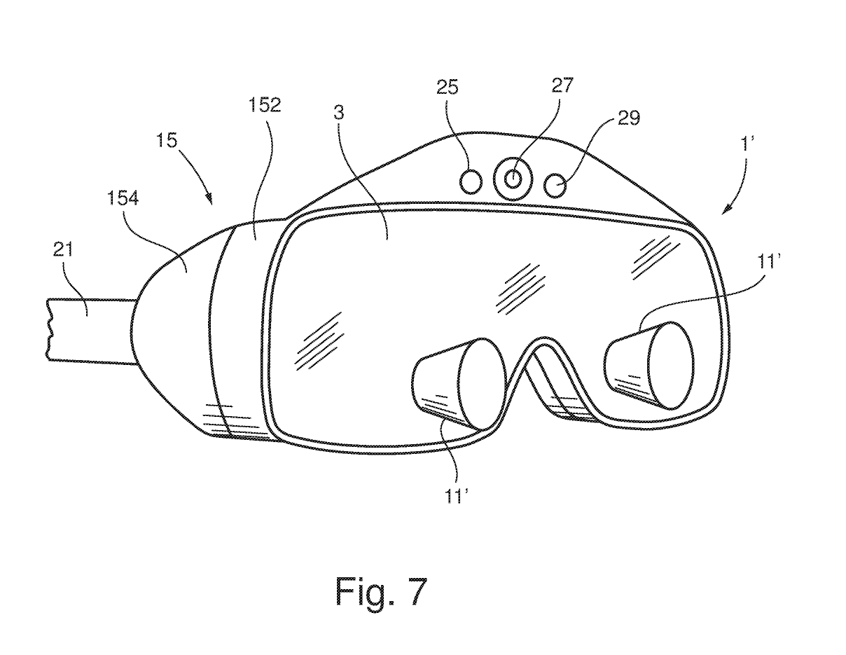

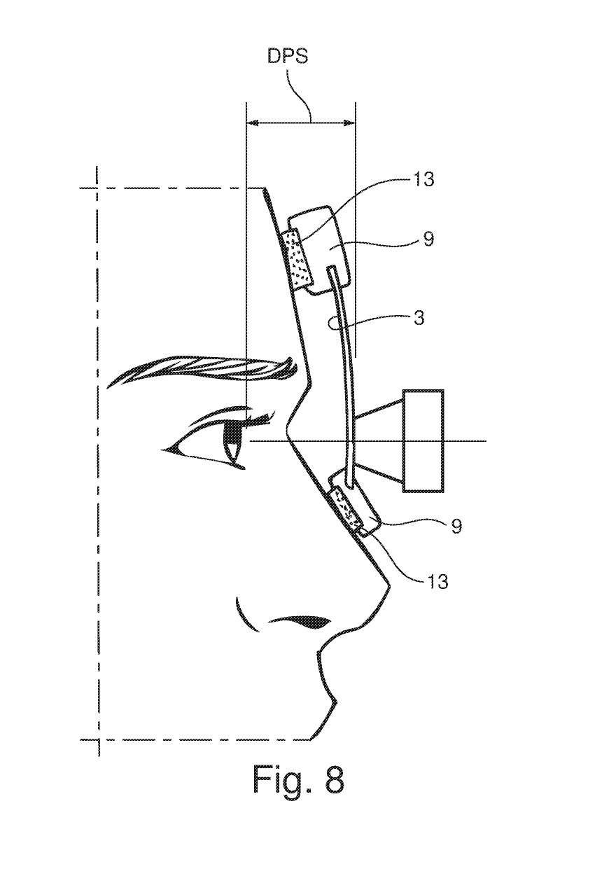

For such a purpose, the mask 1 shape is such to position the screen 3 at a sufficient distance DPS from the surface of the user's eyeballs in such a way as to form a space between the user's face and the internal surface of the screen 3 wide enough to contain normal corrective glasses (FIG. 8).

The distance DPS, measured along a substantially fore-aft direction and perpendicularly to the internal surface of the screen 3, is preferably equal or greater than 1 cm, more preferably equal or greater than 2 cm, more preferably equal or greater than 2.5 cm, more preferably equal or greater than 3 cm and most preferably equal or greater than 5 cm.

The mask 1 can be equipped with a bracket or other support not shown to attach lights, video cameras or microphones if any to respectively illuminate or video record and/or audio record the field of operation or whatever the user is observing.

The said lights can, for example, be punctiform.

The support can be equipped with a rapid attachment system to fix the light, microphone or video camera. Alternatively, the frame 15 can be equipped with a row of punctiform light sources, for example LED, arranged along the frame itself, or with one or more strips of lights which extend over most or the whole length of the frame 15 or around the whole perimeter of the screen 3: light sources distributed in such a way cast fewer shadows on the field of operation or other work space in comparison to, for example, a single punctiform light source.

Alternatively the light or lights 25, the video camera 27 and the microphone 29 can be enclosed in the frame 15′ (FIG. 7).

Preferably the light or lights 25, the video camera 27 and the microphone 29 are situated in correspondence with or in proximity of the sagittal plane of the mask 1′, in such a way that the lighting and the framing of the field of operation are influenced the least possible by the fact that the user may be right or left handed.

The previous description shows how the mask 1 allows very effective avoidance of infections transmitted through the eye, ocular contaminations and any other eye lesion that could happen in the operating room, clinic, medical practice, laboratory or other workplace as a result of splashes, droplets, other projections of infected or contaminated liquid or solid particles as well as infected or contaminated aerosol or incandescent splinters.

The mask 1 is particularly effective in blocking projections that approach the eyes along a trajectory which takes them close or tangent to the surface of the face.

At the same time mask 1 allows a surgeon, an artisan or a scientist, to work while having both a magnified central vision with the necessary magnification power and a good non-magnified and potentially corrected peripheral vision through the screen 3, a peripheral vision that ensures that the user will not lose track of the surroundings of the magnified area on which he has to operate, of the whole field of operation and of the operating room. Without removing the mask the surgeon can see, as normal, the tools and machinery in the room.

The seal 13 and the frame 15 ensure that the mask can be adapted to the facial conformation of each user and discharge the pressure of the mask 1, and in particular the weight of illuminating devices, video cameras, microphones and batteries which may be mounted on the mask itself over a much wider surface area of the user's face as opposed to only the root and ridge of the nose or the helix and the auricle of the ears on which usually the nose pads and temple tips of spectacle frames rest and which are present, for example, in the devices described in publications U.S. Pat. No. 2,226,941, U.S. Pat. No. 4,196,966 o U.S. Pat. No. 5,331,357, making the mask 1 much more comfortable to wear even for long periods of several hours.

Moreover, the seal 13 and the frame 15 make more tolerable the weight of lights, microphones, video cameras and batteries mounted on the mask and which allow the surgeon or dentist to illuminate better the area in which they are operating, or to document surgical operations, providing patients and insurance companies objective evidence of the correctness—or incorrectness—of the operations, and important teaching material for students and trainees.

The previously described embodiments are susceptible to various modification and variations without losing the protective features of the present invention.

For example, a mask may clearly be used not only for medical purposes, but also, for example, for watchmaking or goldsmith work, for artisanal work in general, modelling, mechanical and electronic manufacturing, clockmaking, cosmetics, agronomy and biology and art conservation.

The magnifying optical system 5 can include a monocular system containing a single telescope of Galilean or prismatic type, or magnifying systems differing from the telescope, such as microscopes. Furthermore, the binocular version of the magnifying optical system 5 can be equipped with a system to regulate the interpupillary distance.

The screen 3 can form a single visor, as shown in the attached Figures, or two separate transparent eye guards. Telescopes, microscopes or other magnifying systems 11′ can be hooked, inserted, glued or welded directly onto the screen 3, as shown in FIG. 7. Additionally or alternatively to the aforementioned air vents, the screen 3 can undergo an antifogging chemical treatment.

The seal 13 or the entire mask 1 can be disposable (single use) and the seals 13 easily substituted.

The light can be equipped with an orange/amber filter.

Battery and memory packs can be accommodated inside the frame 15 or hooked onto the support strap of the mask 21.

The whole mask or just some of its components may be disposable.

The support 7 can be equipped with a rapid attachment system to fix the magnifying system 5.

Moreover, all the components can be substituted by technically equivalent parts.

For example the materials used, or the dimensions, can be changed to suit the technical requirements.

Please note that the following sentence “A contains B, C, D” or “A is formed of B, C, D” also includes and describes the specific case in which “A is constituted of B, C, D”.

Examples and lists of possible variants outlined in the present request are to be understood as non exhaustive.

1. Protective mask (1, 1′, 1″) comprising:
a substantially transparent screen (3);
a magnifying optical system (5);
a fixing system (7) keeping the magnifying system in a substantially fixed position relative to the screen (3);
a sealing system (13);
and wherein, when a user (U) wears the mask (1, 1′):
the magnifying system (5) is configured for magnifying the images viewed by the user (U) occupying a first more central portion of the field of view, with a minimum focusing distance equal to or less than 10 meters;
the screen (3) is configured for covering the eyes of the user enabling vision in a second portion of its field of view more peripheral than the first portion of the visual field;
the seal system is configured for resting against the user's face, forming a barrier that surrounds or otherwise extends around the screen and the eyes of the user;
the screen (3) and the sealing system, along with other possible elements of the mask, form a casing that completely encloses and isolates from the external environment the user's eyes and the area of the face adjacent to the eyes, forming a barrier that prevents solid or liquid projections such as, for example, solid fragments, splashes, drops or aerosol particles from penetrating into the mask (1, 1′) and reaching the user's eyes.

2. Mask (1, 1′) according to claim 1, wherein the magnifying system (5) comprises one or more elements of the following group: a prismatic or possibly Galilean telescope, a microscope, a magnifying lens, a monocular optical system, a binocular optical system.

3. Mask (1, 1′) according to claim 1, wherein the magnifying system (5) has a minimum focusing distance less than or equal to 1 meter.

4. Mask (1, 1′) according to claim 1, wherein the magnifying system (5) has a magnifying power between 2 and 20 magnifications.

5. Mask (1, 1′) according to claim 1, wherein the sealing system includes a seal support surface (13) which extends around the screen (3), and is configured for resting against the face of the user along at least 80% of the perimeter of the screen (3) forming a barrier which prevents projections solid or liquid such as, for example, solid fragments, splashes, drops or aerosol particles to penetrate into the mask (1, 1′) and reach the eyes of the user (U).

6. Mask (1, 1′) according to claim 1, in which the seal support (13) is configured for resting against the face of the user along the entire perimeter of the screen (3).

7. Mask (1, 1′) according to claim 1, in which the support seal (13) is configured for resting against the face of the user:
so as to enclose the superciliary arches and/or and the orbits of the user; and/or
so as to pass over the nose without enclosing the user's nostrils inside the mask (1, 1′); and/or
so as to pass over the cheekbones or enclosing them within the mask (1) without enclosing the user's mouth within the mask (1, 1′).

8. Mask (1, 1′) according to claim 1, wherein the screen (3) has an extension such as to allow the field of view of the two eyes of the user to extend, with reference to the anatomical location of the user's body:
5—by at least 120°, more preferably by at least 140° and even more preferably by at least 160°, measured in a transverse plane; and/or by at least 80°, more preferably by at least 100° and even more preferably by at least 120° measured in a sagittal plane.

9. Mask (1, 1′) according to claim 8, wherein the screen (3) extends over an area equal to or greater than about 50 square centimetres, possibly equal to or greater than 80 square centimetres, possibly equal to or greater than 135 square centimetres.

10. Mask (1, 1′) according to claim 8, wherein the screen (3) has an extension such that the minimum distance (DVM) of its peripheral edges from the axis of the pupils of the user measured in a sagittal plane when the user is looking straight ahead, is equal to or greater than about 3 cm.

11. Mask (1, 1′) according to claim 8, wherein the screen (3) has an extension such that the minimum distance (DOM) of the perimeter edges of the screen (3) from the axis of the pupils of the user measured in a transverse plane, when the user (U) is looking straight ahead, is equal to or greater than 5 centimetres.

12. Mask (1, 1′) according to claim 1, comprising one or more air vents (17) allowing the intake and discharge of air in the gap between the mask itself (1, 1′) and the user's face so as to avoid or reduce fogging of the screen (3).

13. Mask (1, 1′) according to claim 12, wherein one or more air vents are provided with one or more filters (19) configured for preventing solid or liquid particles from entering the space between the mask itself (1, 1′) and the face of the user reaching the eyes of the user (U).

14. Mask (1, 1′) according to claim 13, wherein the filters (19) comprise one or more elements selected from the following group: a layer of fibrous or spongy material, a layer of sintered material, a bed of granular material, a labyrinth- or serpentine duct, a duct forming a Z, zigzag or elbow-shaped path.

15. Mask (1, 1′) according to claim 1, comprising one or more of the following elements: a light (25) to illuminate the objects arranged in front of the mask itself; a video camera (27); a microphone (29).

16. Supply kit comprising:
a) a mask (1″) having the features according to of claim 1;
b) an optical corrector (33) itself comprising:
b.1) two lenses (34) with corrective power; and
b.2) a frame (35) comprising a bridge (350);
and the optical corrector (33) configured for hooking, or more generally fixing, onto the mask (1″) in such way that the lenses (34) are each in front of one the user's eyes, and the bridge (350) extends over the user's nose and between the two lenses (34).

17. Supply kit according to claim 1, wherein the optical corrector (33) is configured for being fixed to an area of the mask (1″) corresponding to or in proximity of the user's nose when he/she is normally wearing the mask (1″).
